(19) United States
(12) Patent Application Publication
(10) Pub. No.: US 2024/0060033 A1
(43) Pub. Date: Feb. 22, 2024

(54) System, and a method for Algal Cultivation

(71) Applicant: RELIANCE INDUSTRIES LIMITED, Mumbai (IN)

(72) Inventors: Ajit SAPRE, Tampa, FL (US); Santanu DASGUPTA, Mumbai (IN); Meenakshi Sundaram CHELLIAH, Chennai 28 (IN); Ganesh Lakshman MADDIKERI, Thane (IN); Mohan NATARAJAN, Villupuram district (IN)

(73) Assignee: RELIANCE INDUSTRIES LIMITED, Mumbai (IN)

(21) Appl. No.: 18/451,996

(22) Filed: Aug. 18, 2023

(30) Foreign Application Priority Data
Aug. 19, 2022 (IN) 202221047385

(51) Int. Cl. C12N 1/12; C12M 1/00; C12M 1/36

(52) U.S. Cl. CPC C12N 1/12; C12M 21/02; C12M 23/18; C12M 23/38; C12M 29/00; C12M 27/20; C12M 41/48

(57) ABSTRACT

The present disclosure provides system (10) and a method for algal cultivation. The system for algal cultivation comprises a reservoir (1) which is configured to contain a fluid medium (6) having an algal culture; at least one circulating means (3) which is configured to circulate the fluid medium in the reservoir at a desired velocity; at least one covering means (5) which is configured in its operative configuration to at least partially cover an opening of the reservoir to at least partially block the light incident on the fluid medium and define intermittent light and dark cycle for the fluid medium for a predetermined period of time in the reservoir. Advantageously, the invention disclosed by the present disclosure improves biomass production, minimizes water evaporation in open pond system, avoids photo-inhibition or stress, provides optimum photosynthetic rate, provides efficient utilization of the space, provides efficient generation of resources such as renewable energy.

### FIELD

The present disclosure relates to a system and a method of algal cultivation.

### Definition

As used in the present disclosure, the following terms are generally intended to have the meaning as set forth below, except to the extent that the context in which they are used indicates otherwise.
Photovoltaics (PV): The term “photovoltaics (PV)” refers to a conversion of light into electricity using semi-conducting materials that exhibit the photovoltaic effect.
Photovoltaic effect: The term “photovoltaic effect” refers to a process that generates voltage or electric current in a photovoltaic cell when it is exposed to sunlight.
Footprint area: The term “footprint area” refers to the covering area/working area of a reservoir, that includes the area of the vertical walls, baffles, support, partition, barrier, and the like.
Pulse amplitude modulation (PAM): The term “pulse amplitude modulation (PAM)” refers to an instrument, which is used to measure the maximum quantum fluorescence efficiency of dark-adapted photosystem II.
Fluorescence: The term “fluorescence” is the ability of emission of light by a substance that has absorbed light or other electromagnetic radiation.
Fluorescence ratio (Fv/Fm): The term “fluorescence ratio (Fv/Fm) is an indicator of photosynthetic performance of algae culture, for green algae the fluorescence ratio is generally between 0.5 to 0.8. The lower values indicate stress or photo-inhibition or indicate down-regulation of photo-synthesis. The fluorescence ratio is measured by using pulse amplitude modulated instrument (PAM). It is calculated by using the following formula:
Fv/Fm is calculated by as (Fm−Fo)/Fm, wherein Fm is the maximum fluorescence yield and Fo is minimum fluorescence yield. The fluorescence ratio of Fv/Fm is a normalized ratio created by dividing variable fluorescence (Fv) by maximum fluorescence (Fm).

### BACKGROUND

The background information herein below relates to the present disclosure but is not necessarily prior art.

Conventionally, the algal cells use photo-active radiation (PAR) to drive photo-synthesis machinery and produce biomolecules such as protein, lipid and carbohydrates. In open pond cultivation, when algal cells are exposed to continuous light for a longer duration, the photosynthetic activity is reduced. This reduction in photosynthetic activity is due to reduction in the activity of the light harvesting antenna protein. This reduction can be recovered by exposing algal cells in dark intermittently for certain time. This involves moving algal culture continuously from light to dark zone in day-time for specified time interval. However, there is always a need to improve the cultivation of algae to meet the increasing demand of resources such as renewable energy, biomolecules, high value chemicals, biomass, food, feed and the like.

Therefore, there is felt a need to develop system and method for algal cultivation that overcomes the above-mentioned limitations.

### OBJECTS

Some of the objects of the present disclosure, which at least one embodiment herein satisfies, are as follows.

It is an object of the present disclosure is to ameliorate one or more problems of the background or to at least provide a useful alternative.

An object of the present disclosure is to provide a system for algal cultivation.

Another object of the present disclosure is to provide a system for algal cultivation that utilize a covering means.

Still another object of the present disclosure is to provide a system for algal cultivation that utilize photovoltaic panels as a covering means.

Yet another object of the present disclosure is to provide a system for algal cultivation that can meet the increasing demand of resources such as renewable energy, biomolecule, high value chemicals, biomass, food, feed and the like.

Still another object of the present disclosure is to provide a system for algal cultivation that has enhanced photosynthesis efficiency.

Still another object of the present disclosure is to provide a method for algal cultivation.

Other objects and advantages of the present disclosure will be more apparent from the following description, which is not intended to limit the scope of the present disclosure.

### SUMMARY

The present disclosure relates to a system for algal cultivation. The system for algal cultivation comprises a reservoir which is configured to contain a fluid medium having an algal culture, the reservoir having an opening through which ambient light can be incident on the fluid medium contained in the reservoir in its operative configuration; at least one circulating means which is configured to circulate the fluid medium in the reservoir in its operative configuration at a desired velocity; at least one covering means which is configured in its operative configuration to at least partially cover the opening of the reservoir to at least partially block the light incident on the fluid medium and define intermittent light and dark cycle for the fluid medium for a predetermined period of time in the reservoir to define a predetermined ration of light and dark time.

In an embodiment, the system is automated and comprises motor driven circulating means and motor driven covering means and further comprises the controlling means. The controlling means is configured to regulate the condition for the growth of the algal culture, and comprises:
a memory which is configured to store a set of footprint-determining rules, a set of fluid medium, temperature and level determining rule, light and dark cycle determining rules, velocity-determining rules, and amount of light incident determining rules and predefined instructions; and
a microprocessor which is configured to operate and execute one or more devices of the system, specifically configured to cooperate with the circulating means to define a velocity of the fluid medium in the flow path(s), as well as configured to cooperate with the motorized circulating means and motorized covering means to cover or uncover a desired area in accordance with the exposed area of the reservoir in real-time.

Further, the present disclosure also envisages a method for algal cultivation. The method comprises the following steps of:
providing the reservoir having a flow path and the opening;
filling the reservoir with the fluid medium up to the predetermined depth such that ambient light is incident on the fluid medium contained in the reservoir;
circulating the fluid medium along the flow path at a predetermined velocity;
at least partially covering the opening to at least partially block the light incident on the fluid medium and thereby define intermittent light and dark cycle for the fluid medium for a predetermined period of time in the reservoir to define a predetermined ration of light and dark time incident to which the circulating fluid medium is exposed.

In an embodiment, the fluid medium is circulated at a velocity is in the range of 5 cm/s to 30 cm/s, preferably 5 cm/s to 15 cm/s. The depth of fluid medium is maintained between 5 cm and 30 cm, and the temperature of the fluid medium is maintained between 28° C. and 40° C.

In an embodiment, the method comprises covering the opening with the help of a covering means such that the covered footprint area is in the range of 15% to 55%, preferably in the range of 28% to 40%, still preferably 36%. The light time to dark time ration in the range of 6:1, preferably 3:1.5, and still preferably 1.75:1.

In an embodiment, the method being automated and comprises the following steps of:
(i) sensing, in the reservoir, the depth of the fluid medium, the temperature of the fluid medium, the velocity of the circulating medium in the flow path, and the amount of light incident on the fluid medium by means of a plurality of sensors;
(ii) maintaining the depth of the fluid medium to between 5 cm and 30 cm, maintaining the temperature of the fluid medium in the range of 28 C to 40 C, maintaining the velocity of the circulating medium in the range of 5 cm/s to 30 cm/s, preferably 5 cm/s to 15 cm/s;
(iii) covering the footprint area of the reservoir to the extent of 15% to 55%, preferably in the range of 28% to 40%, still preferably 36%;
(iv) maintaining a light to dark cycle for the fluid medium to lies is in the range of 6:1, preferably 3:1.5, and still preferably 1.75:1 to maintain a florescence ratio in the range of 0.5 to 0.7; and
(v) controlling the sensing, the circulating fluid medium and the operation of the covering by means of a processor and a memory in accordance with predetermined rules.

In an embodiment, the plurality of sensors is selected from a group of sensors consisting of a light sensor, heat sensor, salinity sensor, pH sensor, fluid level sensor, fluid velocity sensor, and cover motion sensor. The sensor is configured to detect the light intensity including low light, moderate light, and intense light as well as the absence of light and rotate said set of solar panels, detect the level of said fluid medium of said reservoir in real-time, as well as the physico-chemical parameters required for the algal culture accordingly.

### BRIEF DESCRIPTION OF THE ACCOMPANYING DRAWING

The system, and the method for algal cultivation, of the present disclosure will now be will now be described with the help of the accompanying drawing, in which:

FIG. 1A illustrate a view of a fully exposed reservoir (algal pond. i.e. no covering means or plastic sheet over the surface of pond or reservoir) in accordance with the first embodiment of the present disclosure;

FIG. 1B illustrates a view of a reservoir (or an algal pond) with 20% of footprint area covered with a covering means in accordance with the first embodiment of the present disclosure;

FIG. 1C illustrates a view of a reservoir (or an algal pond) with 36% of footprint area covered with a covering means in accordance with the first embodiment of the present disclosure;

FIG. 1D illustrates a view of a reservoir (or an algal pond) with 50% of footprint area covered with a covering means in accordance with the first embodiment of the present disclosure;

FIG. 2 illustrates performance of the system for the algal cultivation in the form of a bar chart with percentage area covered (along X-axis) and productivity (g/m²/day) (along Y-axis) in accordance with the present disclosure;

FIG. 3A illustrates a schematic view of a fully exposed or uncovered reservoir (.i.e. control condition) in accordance with the second embodiment of the present disclosure;

FIG. 3B illustrates a schematic view of a reservoir (algal pond) with 20% of footprint area covered with photovoltaic panels in accordance with the second embodiment of the present disclosure;

FIG. 3C illustrates a schematic view of a reservoir (algal pond) with 36% of footprint area covered with photovoltaic panels in accordance with the second embodiment of the present disclosure;

FIG. 3D illustrates a schematic view of a reservoir (algal pond) with 50% of footprint area covered with photovoltaic panels in accordance with the second embodiment of the present disclosure;

FIG. 4A to 4D are corresponding figures of FIG. 3A to FIG. 3D respectively in accordance with the second embodiment of the present disclosure;

### LIST OF REFERENCE NUMERALS

| Reference | |
|---|---|
| no. | Reference |
| 10 | System for algal cultivation |
| 1 | Reservoir |
| 2 | Partition wall |
| 3 | Circulating means |
| 4 | Flow path |
| 5 | Covering means |
| 6 | Fluid medium |

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, elements, modules, units and/or components, but do not forbid the presence or addition of one or more other features, elements, components, and/or groups thereof.

As used herein, the term “and/or” includes any and all combinations of one or more of the associated listed elements.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Conventionally, the algal cells use photo-active radiation (PAR) to drive photo-synthesis machinery and produce biomolecules such as protein, lipid and carbohydrates. In open pond cultivation, when algal cells are exposed to continuous light for a longer duration, the photosynthetic activity is reduced. This reduction in photosynthetic activity is due to reduction in the activity of the light harvesting antenna. This reduction can be recovered by exposing algal cells in dark intermittently for certain time. This involves moving algal culture continuously from light zone to dark zone in daytime for specified time interval. However, there is always a need to improve the cultivation of algae to meet the increasing demand of renewable energy.

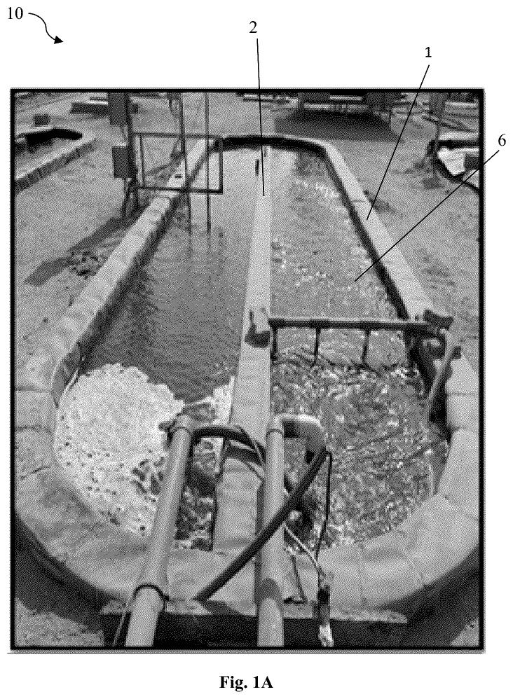

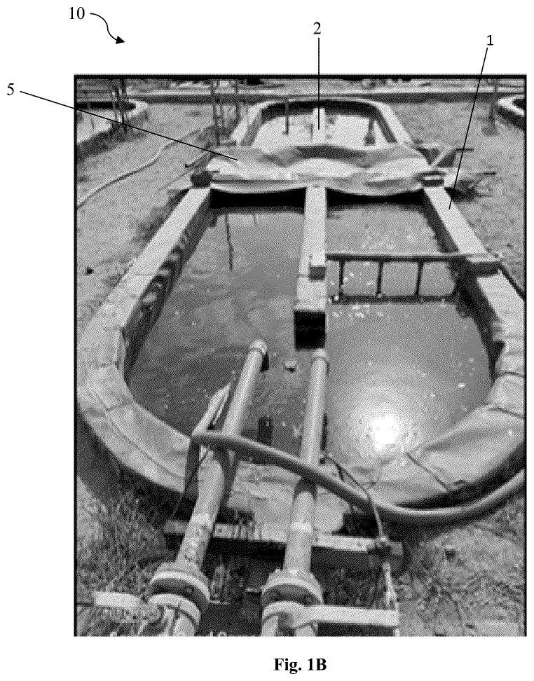

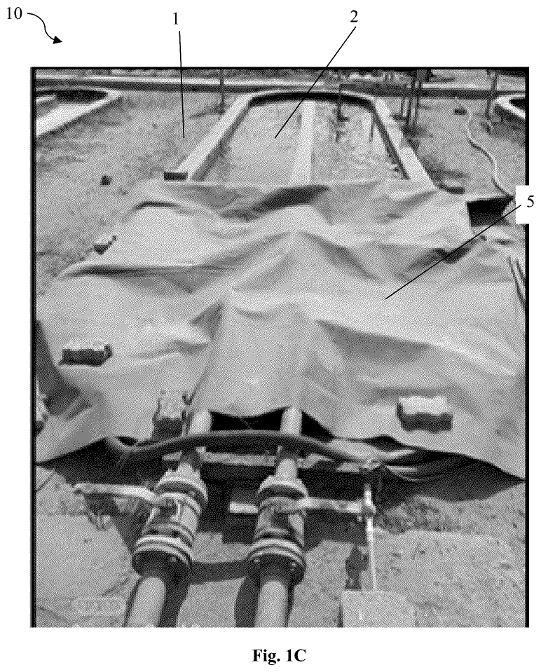

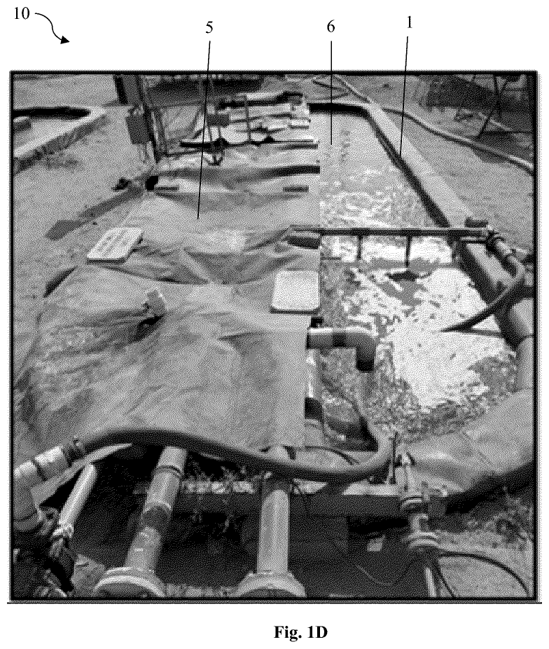

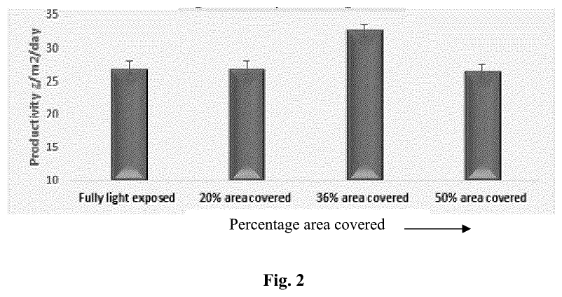

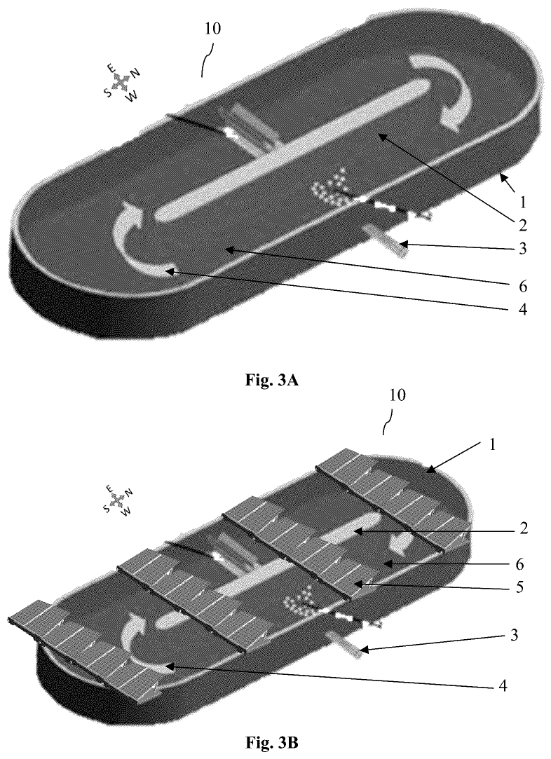

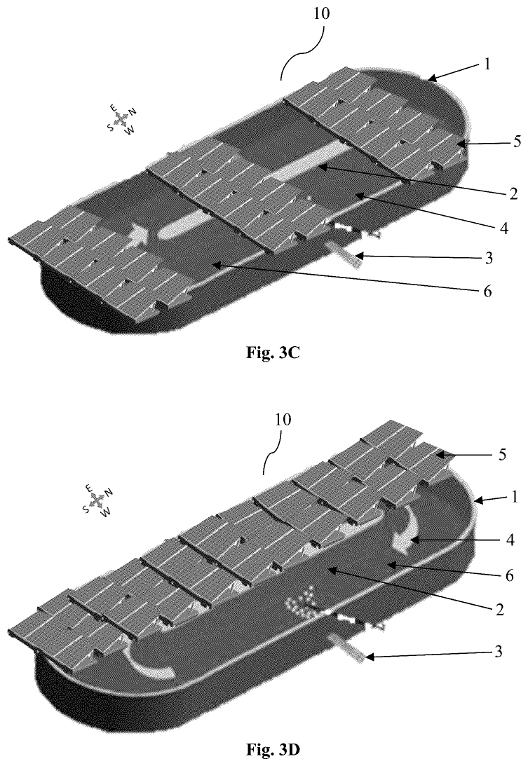

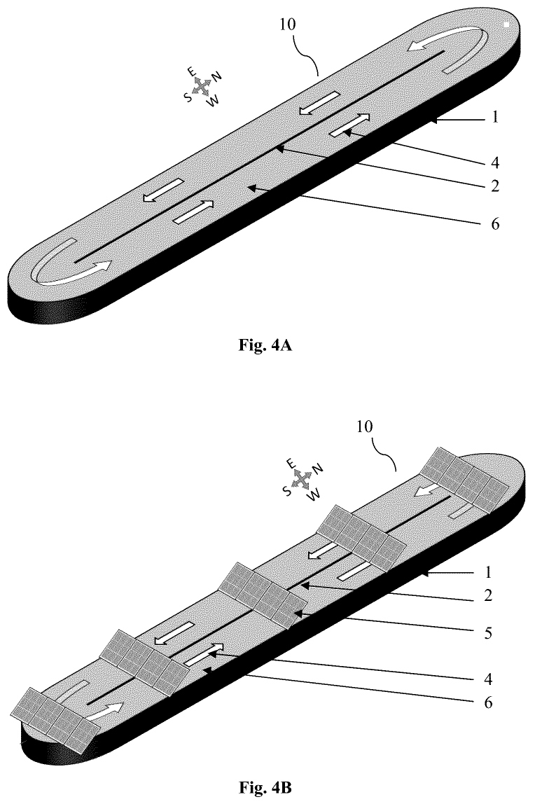

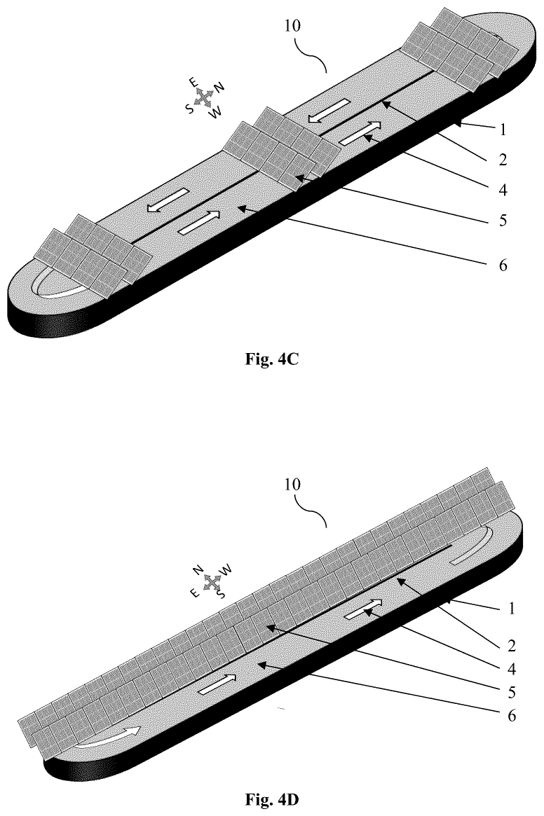

In order to address the limitations of conventional algal cultivation, a system (10), and a method for algal cultivation that overcomes the above discussed problems is developed. The different embodiments of the present disclosure are explained with reference to FIG. 1A-FIG. 1D, FIG. 2, FIG. 3A-FIG. 3D, and FIG. 4A-FIG. 4D.

In an aspect, the present disclosure provides a system (10) for algal cultivation.

The system (10) for algal cultivation comprises a reservoir (1), at least one circulating means (3) and at least one covering means (5). The reservoir (1) is containing a fluid medium (6) with an algal culture. The reservoir (1) has an opening through which ambient light can be incident on the fluid medium contained in the reservoir (1) in its operative configuration. The circulating means (3) is configured to circulate the fluid medium in the reservoir (1) in its operative configuration at a desired velocity. The covering means (5) is configured in its operative configuration to at least partially cover the opening of the reservoir (1) to at least partially block the light incident on the fluid medium and define intermittent light and dark cycle for the fluid medium for a predetermined period of time in the reservoir (1) to define a predetermined ration of light and dark time.

In accordance with the present disclosure, the reservoir (1) is selected from the group consisting of a pond, an open raceway pond, and an open pond. In an embodiment, the reservoir (1) is an open raceway pond. In another embodiment, the reservoir (1) is an open pond.

In a preferred embodiment, the reservoir (1) is the open raceway pond, generally oval-shaped with curved end walls and straight side walls. A partition wall (2) is provided in the reservoir (1) spaced apart from the side walls to define a path for the fluid medium (6) circulating in the reservoir in its operative configuration. The side walls of the reservoir (1) being at least partially embedded in the ground or projecting therefrom. The side walls and the partition of the reservoir are aligned in a direction dependent upon the location of the reservoir. The partition walls are parallel to each other.

In a preferred embodiment, the direction of flow of the fluid medium is north-south direction. However, the direction of flow of the fluid medium in the reservoir depends on the geographical condition of the country.

In an embodiment, the depth of the reservoir (1) is in the range of 5 cm and 30 cm. In an exemplary embodiment, the predetermined depth of the fluid medium (6) having the algal culture is 10 cm.

The temperature of the fluid medium is controlled to be in the range of 28° C. to 40° C.

In accordance with the present disclosure, the fluid medium (6) is selected from the group consisting of water, seawater, and a minimal medium.

In accordance with the present disclosure, the algae can include, but not limited to green algae, blue green algae, and non-motile green algae. Other algae can also be used.

In accordance with the present disclosure, the predetermined depth of the fluid medium (6) having the algal culture in the reservoir (1) is maintained by using a level sensor and a pumping means.

In an embodiment, the partition wall (2) is prepared from a material selected from the group consisting of masonry, clay, plastic, wood, metal and fibrous material.

In an embodiment, the flow path/s (4) are defined based on the culture depth, flow velocity, pond area, light time and dark time.

Further, the circulating means is configured to circulate the fluid medium along the flow path at a desired velocity. The circulating means being defined by a pump or a pump and paddle wheels.

In an exemplary embodiment, the circulating means (3) is a pump.

The circulating means (3) is used for flow circulation and maintaining the desired flow velocity of the algal culture and for mixing the algal culture. In accordance with the present disclosure, the pump flow rate and head is chosen to maintain desired flow velocity.

In accordance with an embodiment, the circulating means (3) is paddle wheel, which requires higher depth of the fluid medium.

In accordance with the present disclosure, the predetermined velocity is in the range of 5 cm/s to 15 cm/s. In an embodiment, the predetermined velocity is in the range of 5 cm/s to 10 cm/s. In another embodiment, the predetermined velocity is in the range of 10 cm/s to 15 cm/s. In an exemplary embodiment, the predetermined velocity is 10 cm/s.

In accordance with present disclosure, the culture depth (or overall pond volume) affects the velocity of the fluid medium (6) having algal culture.

In accordance with an embodiment, it is desirable to use the working height of the reservoir and the channeling means is substantially equivalent to the depth of the fluid medium.

Further, the covering means (5) is configured to cover a footprint area of the reservoir (1). The covering means (5) being aligned in a direction selected between the side walls and across the flow path, parallel to the flow path and partially parallel to the flow path. The covering means (5) is selected from a group consisting of removable covering means, photovoltaic panel, wooden block, plastic sheet, cloth, and permanent construction.

In an exemplary embodiment, the covering means (5) is a plastic sheet. In another exemplary embodiment, the covering means (5) are photovoltaic panels.

In accordance with the present disclosure, the predetermined covered footprint area is in the range of 15% to 55%. In an embodiment, the predetermined covered footprint area is in the range of 15% to 28%. In another embodiment, the predetermined covered footprint area is in the range of 28% to 40%. In still another embodiment, the predetermined covered footprint area is in the range of 40% to 55%. In an exemplary embodiment, the predetermined covered footprint area is 20%. In another exemplary embodiment, the predetermined covered footprint area is 37%. In still another exemplary embodiment, the predetermined covered footprint area is 50%.

In a preferred embodiment, the covering means (5) are a plurality of movable photovoltaic panels. The movement of the photovoltaic panels is configured to cover and uncover the opening of the reservoir. The actuation of the photovoltaic panels is being controlled by means of at least one first motor, and the actuation of the circulating means (3) is controlled by means of at least one second motor. The first motor and the second motor being powered by the power obtained from the photovoltaic panels. The photovoltaic panels powers the circulating means or the movement of the covering means in the operative configuration of the system.

In accordance with an embodiment of the present disclosure, the covering means (5) is disposed along the shorter axis of the reservoir (1). In accordance with another embodiment of the present disclosure, the covering means (5) is disposed along the longer axis of the reservoir (1).

In accordance with an embodiment of the present disclosure, the reservoir (1) is covered by using a covering means (5) to provide the dark time required for algal cultivation.

In an embodiment, the orientation of the photovoltaic panel is in east-west direction along the width of the photovoltaic panel.

In accordance with an embodiment of the present disclosure, the reservoir (1) is covered by using the photovoltaic panel to provide the dark time required for algal cultivation.

An advantage of integrating algae cultivation with photovoltaic panel is that a double energy can be harvested from the given area.

In accordance with the present disclosure, the predetermined light time to dark time ratio is in the range of 6:1. In an embodiment, the predetermined light time to dark time ratio is in the range of 1.5:1. In another embodiment, the predetermined light time to dark time ratio is in the range of 3:1 to 1.5:1. In still another embodiment the predetermined light time to dark time ratio is in the range of to 6:1. In an exemplary embodiment, the predetermined light time to dark time ratio is 1.75:1. In another exemplary embodiment, the predetermined light time to dark time ratio is 1.00:1.00. In still another exemplary embodiment, the predetermined light time to dark time ratio is 4.50:1.

In accordance with the present disclosure, the light time is in the range of 80 seconds to 145 seconds. In an embodiment, the light time is in the range of 80 seconds to 95 second. In another embodiment, the light time is in the range of 95 seconds to 120 second. In still another embodiment, the light time is in the range of 120 seconds to 150 seconds. In an exemplary embodiment, the light time is 140 seconds. In another exemplary embodiment, the light time is 90 seconds. In still another exemplary embodiment, the light time is 110 seconds.

In accordance with the present disclosure, the dark time is in the range of 15 seconds to 120 seconds. In an embodiment, the dark time is in the range of 15 seconds to 40 seconds. In another embodiment, the dark time is in the range of 40 seconds to 90 seconds. In still another embodiment, the dark time is in the range of 90 seconds to 120 seconds. In an exemplary embodiment, the dark time is 80 seconds. In another exemplary embodiment, the dark time is 20 seconds. In still another exemplary embodiment, the dark time is 110 seconds.

In accordance with the present disclosure, the dark time of the algal culture in the reservoir (1) is provided by covering the footprint area of the reservoir (1).

In accordance with an embodiment of the present disclosure, temperature of the algal culture is in the range of 28° C. to 40° C.

In accordance with the present disclosure, the fluorescence ratio (Fv/Fm) is in the range of 0.5 to 0.7. In an exemplary embodiment, the fluorescence ratio (Fv/Fm) is 0.58±0.05. In another exemplary embodiment, the fluorescence ratio (Fv/Fm) is 0.60±0.03. In still another exemplary embodiment, the fluorescence ratio (Fv/Fm) is 0.59±0.07.

In accordance with the present disclosure, the system of the present disclosure enhances algal productivity when up to 36% of footprint area of the reservoir (1) is covered.

In accordance with an embodiment of the present disclosure, there is a possibility of using the covered footprint area of the reservoir (1) by using photovoltaic/solar panels to generate sustainable power. This sustainable power can be used to operate and generate income of the excess power produced through this integrated cultivation.

In accordance with the present disclosure, the dark time can be provided by designing the reservoir (1) in such way that it can provide the required light and dark time.

In accordance with an embodiment of the present disclosure, the system for algal cultivation integrates photovoltaic in algae cultivation for power generation.

The power generated by photovoltaic panels is converted to artificial light energy or other form of energy, and may be utilized by the algal culture of the system of the present disclosure. The additional power generated by photovoltaic panels can be stored in battery.

Further, the system includes a plurality of sensors. The sensor is configured to sense: i) the depth of the fluid medium in the reservoir, ii) the temperature of the fluid medium in the reservoir, iii) the velocity of the fluid medium in the flow path iv) the amount of light incident on the fluid medium in the reservoir. The system also includes a power device for supplying the power to the sensors. The power device being at least one selected from a group consisting of external power device. The power device is mounted on the covering means, and being the part of the covering means and a combination of power devices for powering the sensors, the circulating means, and the covering means.

In an embodiment, the plurality of sensors selected from a group of sensors consisting of a light sensor, heat sensor, salinity sensor, pH sensor, fluid level sensor, fluid velocity sensor, and cover motion sensor. The plurality of sensors is configured to detect the light intensity including low light, moderate light, and intense light as well as the absence of light and rotate the set of solar panels, detect the level of said fluid medium of the reservoir (102) in real-time, as well as the physico-chemical parameters required for the algal culture accordingly.

Further, the system (10) is automated and comprises the motor driven circulating means and the motor driven covering means and further comprises the controlling means which is configured to regulate the condition for the growth of the algal culture. The controlling means comprises
a memory (not shown) which is configured to store a set of footprint-determining rules, a set of fluid medium, temperature and level determining rule, light and dark cycle determining rules, velocity-determining rules, and amount of light incident determining rules and predefined instructions; and
a microprocessor (not shown) which is configured to operate and execute one or more devices of the system (10), specifically configured to cooperate with the circulating means (3) to define a velocity of the fluid medium in the flow path(s), as well as configured to cooperate with the motorized circulating means and motorized covering means to cover or uncover a desired area in accordance with the exposed area of the reservoir (1) in real-time.

In an embodiment, the system (10) is controlled and operated remotely over a wireless communication network that consists of the Internet of Things (IoT), short-range communication network, and long-range communication network.

The system (10) for algal cultivation of the present disclosure minimizes the algal stress i.e. photo-inhibition.

The system for algal cultivation of the present disclosure minimizes the water evaporation and prevents the rapid salinity increase in the reservoir (1).

In accordance with the present disclosure, the system (10) for algal cultivation provides optimum photosynthetic conditions to algal cells.

The system of the present disclosure has enhanced photosynthetic efficiency and subsequently has higher biomass production.

The system (10) for algal cultivation of the present disclosure can reduce the effect of variation in temperature and salinity while operating pond at the shallow depth by using circulating means (3), covering means (5) and maintaining the predetermined depth.

Further, the present disclosure also envisages a method for algal cultivation. The method comprises the following steps of:
providing the reservoir (1) with a flow path and the opening;
filling the reservoir (1) with the fluid medium up to a predetermined depth such that ambient light is incident on the fluid medium contained in the reservoir (1);
circulating the fluid medium along the flow path at a predetermined velocity;
at least partially covering the opening to at least partially block the light incident on the fluid medium and thereby define intermittent light and dark cycle for the fluid medium for a predetermined period of time in the reservoir (102) to define a predetermined ration of light and dark time incident to which the circulating fluid medium is exposed.

A reservoir (1) having at least one partition wall (2) defining flow paths (4) is established.

A fluid medium (6) having an algal culture of predetermined depth is added in a reservoir (1).

In an embodiment, the fluid medium is circulated at a velocity is in the range of 5 cm/s to 30 cm/s, preferably 5 cm/s to 15 cm/s, the depth of fluid medium is maintained between 5 cm and 30 cm, the temperature of the fluid medium is maintained between 28° C. and 40° C.

In accordance with the present disclosure, the predetermined depth of fluid medium (6) having the algal culture in the reservoir (1) is in the range of 5 cm to 30 cm. In the exemplary embodiments, the predetermined depth of the fluid medium (6) having the algal culture is 10 cm.

The method further includes covering the opening with the help of a covering means such that the covered footprint area.

In accordance with the present disclosure, the covered predetermined area is in the range of 15% to 55%. In an embodiment, the predetermined covered area is in the range of 15% to 28%. In another embodiment, the predetermined covered area is in the range of 28% to 40%. In still another embodiment, the predetermined covered area is in the range of 40% to 55%. In an exemplary embodiment, the covered predetermined area is 20%. In another exemplary embodiment, the covered predetermined area is 37%. In still another exemplary embodiment, the covered area is 50%.

In accordance with an embodiment of the present disclosure, the covering means (5) is disposed along the shorter axis of the reservoir (1). In accordance with another embodiment of the present disclosure, the covering means (5) is disposed along the longer axis of the reservoir (1).

The fluid medium (6) having the algal culture is circulated at a predetermined velocity through flow path/s (4) of the reservoir (1) to expose the algal culture to an intermittent light time and dark time to obtain the algal culture having high biomass.

In accordance with the present disclosure, the predetermined velocity is in the range of 5 cm/s to 30 cm/s. In an embodiment, the predetermined velocity is in the range of 5 cm/s to 10 cm/s. In another embodiment, the predetermined velocity is in the range of 10 cm/s to 15 cm/s. In still another embodiment, the predetermined velocity is in the range of 15 cm/s to 30 cm/s. In an exemplary embodiment, the predetermined velocity is 10 cm/s.

In accordance with the present disclosure, the light time to dark time ratio is in the range of 6:1. In an embodiment, the predetermined light time to dark time ratio is in the range of 1.50:1. In another embodiment, the predetermined light time to dark time ratio is in the range of 1.50:1 to 3:1. In still another embodiment the predetermined light time to dark time ratio is in the range of 3:1 to 6:1. In an exemplary embodiment, the predetermined light time to dark time ratio is 1.75:1. In another exemplary embodiment, the predetermined light time to dark time ratio is 1.00:1.00. In still another exemplary embodiment, the predetermined light time to dark time ratio is 4.50:1.

In accordance with the present disclosure, the light time is in the range of 80 seconds to 145 seconds. In an embodiment, the light time is in the range of 80 seconds to 90 second. In another embodiment, the light time is in the range of 90 seconds to 120 second. In still another embodiment, the light time is in the range of 120 seconds to 145 seconds. In an exemplary embodiment, the light time is 135 seconds. In another exemplary embodiment, the light time is 86 seconds. In still another exemplary embodiment, the light time is 105 seconds.

In accordance with the present disclosure, the dark time is in the range of 15 seconds to 120 seconds. In an embodiment, the dark time is in the range of 15 seconds to 40 seconds. In another embodiment, the dark time is in the range of 40 seconds to 80 seconds. In still another embodiment, the dark time is in the range of 80 seconds to 120 seconds. In an exemplary embodiment, the dark time is 75 seconds. In another exemplary embodiment, the dark time is 19 seconds. In still another exemplary embodiment, the dark time is 105 seconds.

In accordance with the present disclosure, the predetermined light time to dark time ratio is in the range of 6:1. In an embodiment, the predetermined light time to dark time ratio is in the range of 1.50:1. In another embodiment, the predetermined light time to dark time ratio is in the range of 1.50:1 to 3:1. In still another embodiment the predetermined light time to dark time ratio is in the range of 3:1 to 6:1. In an exemplary embodiment, the predetermined light time to dark time ratio is 1.75:1. In another exemplary embodiment, the predetermined light time to dark time ratio is 1.00. In still another exemplary embodiment, the predetermined light time to dark time ratio is 4.50:1.

In accordance with an embodiment of the present disclosure, temperature of the fluid medium having the algal culture is in the range of 28° C. to 40° C.

In accordance with the present disclosure, the Fv/Fm is in the range of 0.5 to 0.7. In an exemplary embodiment, the Fv/Fm is 0.58±0.05. In another exemplary embodiment, the Fv/Fm is 0.60±0.03. In still another exemplary embodiment, the Fv/Fm is 0.59±0.07.

Further, the method being automated and further comprises the following steps of:
i. sensing, in the reservoir (1), the depth of the fluid medium, the temperature of the fluid medium, the velocity of the circulating medium in the flow path, and the amount of light incident on the fluid medium by means of a plurality of sensors;
ii. maintaining the depth of the fluid medium to between 5 cm and 30 cm, maintaining the temperature of the fluid medium in the range of 28 C to 40 C, maintaining the velocity of the circulating medium in the range of 5 cm/s to 30 cm/s, preferably 5 cm/s to 15 cm/s;
iii. covering the footprint area of the reservoir (1) to the extent of 15% to 55%, preferably in the range of 28% to 40%, still preferably 36%;
iv. maintaining a light to dark cycle for the fluid medium to lies is in the range of 6:1, preferably 3:1.5, and still preferably 1.75:1 to maintain a florescence ratio in the range of 0.5 to 0.7; and
v. controlling the sensing, the circulating fluid medium and the operation of the covering by means of a processor and a memory in accordance with predetermined rules.

In an embodiment, the method is controlled remotely with the help of a wireless communication network that consists of the Internet of Things (IoT), short-range communication network, and long-range communication network.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further illustrated herein below with the help of the following experiments. The experiments used herein are intended merely to facilitate an understanding of the ways in which the embodiments herein may be practiced and to further enable those of skill in the art to practice the embodiments herein. Accordingly, the experiments should not be construed as limiting the scope of embodiments herein. These laboratory scale experiments can be scaled up to industrial/commercial scale and the results obtained can be extrapolated to industrial/commercial scale.

### EXPERIMENTAL DETAILS

### Example 1

To demonstrate the effect of covering the footprint area of an algal pond, a system was developed with in accordance with four conditions. In all the four conditions, the footprint area of the reservoir (pond) was varied by means of the covering means (5) or plastic sheet to predict the range of effective footprint area to obtain the algal culture with high biomass. The system was pump driven and 21 m² open raceway ponds at shallow depth cultivation.

In first condition (Control condition), there was no covering means (5) or plastic sheet over the surface of pond or reservoir (1) (Zero % coverage of footprint area) .i.e. fully light exposed pond as shown in FIG. 1A. In second condition, the covering means covers, 20% (approx.) of the footprint area by disposing the covering means or the plastic sheet along the shorter axis of the algal pond as shown in FIG. 1B. In third condition, the covering means covers 36.4% of the footprint area by disposing the covering means or the plastic sheet along the shorter axis of the algal pond as shown in FIG. 1C. And, In fourth condition, the covering means covers 50% of the footprint area by disposing the covering means or the plastic sheet along the longer axis of the algal pond as shown in FIG. 1D. For each condition, a variable process parameter such as Light time, dark time, culture velocity was selected to obtain the algal culture with high biomass.

It is observed that, for the first condition i.e. control condition, the areal productivity of the biomass was 19 g/m²/day. However, for the second condition, .i.e. 18.2% of the footprint area coverage, the areal productivity of the biomass was 17 g/m²/day; for the third condition, .i.e. 36.4% of the footprint area coverage, the areal productivity of the biomass was 23 g/m²/day; and for the fourth condition, .i.e. 50% of the footprint area coverage, the areal productivity of the biomass was 18 g/m²/day. Table 1 illustrates the effect of covering the footprint area of an algal pond over the areal productivity of the biomass.

TABLE 1
illustrates the effect covering of the footprint area of an algal pond over
the areal productivity of the biomass

| | | | |
|---|---|---|---|
| Pond Details | | | |
| Total pond area | 21 m² | 21 m² | 21 m² |
| Mixing device | Pump | Pump | Pump |
| Culture depth | 10 cm | 10 cm | 10 cm |
| Covering Specification | | | |
| Dark (covered with plastic sheet) | 2 m | 4 m | 11 m |
| Light (open space) | 9 m | 7 m | 11 m |
| Covered footprint area | 18.2% | 36.4% | 50.0% |
| Flow Conditions | | | |
| Culture velocity | 10 cm/s | 10 cm/s | 10 cm/s |
| Light time | 90 sec | 140 sec | 110 sec |
| Dark time | 20 sec | 80 sec | 110 sec |
| Light time/dark time ratio | 4.50 | 1.75 | 1.00 |
| Areal Productivity | | | |
| Experimental | 17 g/m²/d | 23 g/m²/d | 18 g/m²/d |
| Control | 19 g/m²/d | 19 g/m²/d | 19 g/m²/d |

From the above table 1, the pond with 36% of footprint area covered with plastic sheet delivered 21% higher biomass productivity than the fully light exposed pond. This confirms that the intermittent dark time is required to recover light harvesting antenna protein.

Further, when the surface of the pond is exposed to variable light time and dark time, there is average change in optical density of the pond as well as change in average culture temperature. Table 2 illustrates the average optical density change per day after partially covering the algal ponds by means of the covering means.

Table 2 below illustrates the average optical density (O.D.) change per day after partially covering the algal ponds with 20%, 36% and 50% of the footprint area.

TABLE 2
illustrates the average optical density change per day after partially
covering the algal ponds

| | | Measured average | | Fluorescence |
|---|---|---|---|---|
| Covered | Average | culture temperature | | ratio |
| pond area | productivity | Min. | Max. | (Fv/Fm) |
| Control | 19 | 31 | 39 | 0.58 |
| 2 m (~20%) | 17 | 30 | 38 | 0.58 |
| 4 m (~36%) | 23 | 30 | 38 | 0.60 |
| 11 m (50%) | 18 | 31 | 38 | 0.59 |

*Pump driven ponds at 10 cm depth, OD: optical density

It is observed from Table 2 that there was an enhancement in biomass productivity with 36% footprint area covered, as well as there was no biomass loss even at 50% footprint area covered when compared to control (fully light exposed) as well as 20% foot print area covered. There was increase in 21% biomass productivity at 36% footprint area covered as compared to fully light exposed pond.

FIG. 2 illustrates performance of the system for the algal cultivation in accordance with all four conditions as illustrated by FIG. 1A-FIG. 1D. It was observed that the intermittent light and dark cycle by covering the footprint area of the algal pond improved biomass productivity.

### Example 2

The footprint area of the algal pond was covered with a plurality of photovoltaic panels, arranged over the operative surface of the pond. The orientation of the photovoltaic panel was in east-west direction along the width of the photovoltaic panels. The photovoltaic panels were disposed along the shorter axis of the algal pond to cover variable footprint area .i.e. first fully open condition (control) as shown in FIG. 3A and FIG. 4A as similar to the FIG. 1A; second condition, 20% of footprint area was covered by arranging the plurality of photovoltaic panels over the surface of pond as shown in FIG. 3B and FIG. 4B. The photovoltaic panels were disposed along the shorter axis of the algal pond. In third condition, 36% of footprint area was covered by arranging the plurality of photovoltaic panels over the surface of pond as shown in FIG. 3C and FIG. 4C. And, in fourth condition, the photovoltaic panels were disposed along the longer axis of the algal pond to cover 50% of footprint area as shown in FIG. 3D and FIG. 4D.

From the above experimentation, it is concluded that the system for algal cultivation when covered with photovoltaic panels with 36% of footprint area covered was able to produce 97.1 kg of biomass and generated 1517 kWhr of electrical energy. The system for algal cultivation when 36% covered with photovoltaic panels was able to produce 2279 GJ energy per acre of land.

The biochemical composition of the biomass so obtained was 40% protein, 20% total lipid, and 40% carbohydrate. It also consisted of cellulose starch, glycol protein and pigments.

### TECHNICAL ADVANCES AND ECONOMICAL SIGNIFICANCE

The present disclosure described herein above has several technical advantages including, but is not limited to, the realization of the system and method for algal cultivation, that
improves biomass production;
minimizes water evaporation in open pond system;
avoids photo-inhibition or stress because of optimum light and dark cycle
provides optimum photosynthetic rate;
provides efficient utilization of the space;
maintains high biomass during changing temperature and salinity;
provides efficient generation of resources such as renewable energy, biomolecules, high value chemicals, biomass, food, feed and the like;
provide advantages of integrating algae cultivation with photovoltaic panel (PV) and harvesting double energy from the given area; and
provides power generation feasibility for self-sustainability.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or are common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

One of the objects of the Patent Law is to provide protection to new technologies in all fields and domain of technologies. The new technologies shall or may contribute in the country economy growth by way of involvement of new efficient and quality method or product manufacturing in India.

To provide the protection of new technologies by patenting the product or process will contribute significant for innovation development in the country. Further by granting patent the patentee can contribute in manufacturing the new product or new process of manufacturing by himself or by technology collaboration or through the licensing.

The applicant submits that the present disclosure will contribute in country economy, which is one of the purposes to enact the Patents Act, 1970. The product in accordance with present invention will be in great demand in country and worldwide due to novel technical features of a present invention is a technical advancement in the area of sustainable energy. The technology in accordance with present disclosure will provide product cheaper, saving in time of total process of manufacturing. The saving in production time will improve the productivity, and cost cutting of the product, which will directly contribute to economy of the country.

The economy significance details requirement may be called during the examination. Only after filing of this patent application, the applicant can work publically related to present disclosure product/process/method. The applicant will disclose all the details related to the economic significance contribution after the protection of invention.

We claim:

1. A system (10) for algal cultivation, said system (10) comprising:
a reservoir (1) configured to contain a fluid medium having an algal culture, said reservoir (1) having an opening (not shown) through which ambient light can be incident on the fluid medium contained in the reservoir (1) in its operative configuration;
at least one circulating means (3) configured to circulate the fluid medium in said reservoir (1) in its operative configuration at a desired velocity; and
at least one covering means (5) configured in its operative configuration to at least partially cover said opening of the reservoir (1) to at least partially block the light incident on the fluid medium and define intermittent light and dark cycle for the fluid medium for a predetermined period of time in the reservoir (1) to define a predetermined ration of light and dark time.

2. The system (10) as claimed in claim 1, wherein said reservoir (1) is an open raceway pond, generally oval-shaped with curved end walls and straight side walls and a partition wall (2) provided in the reservoir (1) spaced apart from the side walls to define a path for the fluid medium circulating in the reservoir in its operative configuration, said walls of said reservoir (1) being at least partially embedded in the ground or projecting therefrom.

3. The system (10) as claimed in claim 1, wherein the side walls and the partition (2) of the reservoir (1) are aligned in a direction dependent upon the location of the reservoir (1), the partition walls (2) are parallel to each other, the depth of said reservoir (1) is in the range of 5 cm and 30 cm, and the temperature of the fluid medium is controlled to be in the range of 28° C. to 40° C.

4. The system (10) as claimed in claim 1, wherein said circulating means (3) is configured to circulate the fluid medium along the flow path at a velocity in the range of 5 cm/s to 30 cm/s, preferably 5 cm/s to 15 cm/s and said circulating means being defined by a pump or a pump and paddle wheels.

5. The system (10) as claimed in claim 1, wherein said covering means (5) is selected from a group consisting of removable covering means, photovoltaic panel, wooden block, plastic sheet, cloth, and permanent construction, and is configured to cover a footprint area of said reservoir to the extent of 15% to 55%, preferably in the range of 28% to 40%, still preferably 36%, said covering means being aligned in a direction selected between the side walls and across the flow path, parallel to the flow path and partially parallel to the flow path.

6. The system (10) as claimed in claim 5, wherein said covering means (5) are a plurality of movable photovoltaic panels and the movement of said photovoltaic panels to cover and uncover the opening of the reservoir (1) is controlled by means of at least one first motor, and the circulating means (3) is controlled by means of at least one second motor, said first motor and said second motor being powered by the power obtained from said photovoltaic panels.

7. The system (10) as claimed in claim 1, wherein said system includes photovoltaic panels to power said circulating means (3) or the movement of said covering means (5).

8. The system (10) as claimed in claim 1, wherein said system includes circulating means (3), covering means (5), and a plurality of sensors for sensing: i) the depth of the fluid medium in said reservoir (1), ii) the temperature of the fluid medium in said reservoir (1), iii) the velocity of the fluid medium in the flow path iv) the amount of light incident on the fluid medium in said reservoir (1), and a power device for supplying power to said sensors, said power device being at least one selected from a group consisting of external power device, a power device mounted on said covering means, said power device being the part of said covering means and a combination of power devices for powering said sensors, said circulating means (3), and said covering means (5).

9. The system (10) as claimed in claim 1, further comprises a plurality of sensors (116) selected from a group of sensors consisting of a light sensor, heat sensor, salinity sensor, pH sensor, fluid level sensor, fluid velocity sensor, and cover motion sensor configured to detect the light intensity including low light, moderate light, and intense light as well as the absence of light and rotate said set of solar panels, detect the level of said fluid medium of said reservoir (102) in real-time, as well as the physico-chemical parameters required for the algal culture accordingly.

10. The system (10) as claimed in claim 1, wherein said predetermined ration of light and dark time is in the range of 6:1, preferably 3:1.5, and still preferably 1.75:1 to maintain a florescence ratio in the range of 0.5 to 0.7.

11. The system (10) as claimed in claim 1, wherein said system is automated and comprises motor driven circulating means (3) and motor driven covering means (5) and further comprises said controlling means (not shown) configured to regulate the condition for the growth of the algal culture, said controlling means comprising:
i. a memory, configured to store a set of footprint-determining rules, a set of fluid medium, temperature and level determining rule, light and dark cycle determining rules, velocity-determining rules, and amount of light incident determining rules and predefined instructions; and
ii. a microprocessor, configured to operate and execute one or more devices of said system, specifically configured to cooperate with said circulating means to define a velocity of the fluid medium in the flow path(s), as well as configured to cooperate with said motorized circulating means and motorized covering means to cover or uncover a desired area in accordance with the exposed area of said reservoir in real-time.

12. The system (10) as claimed in claim 1, wherein said system (10) is controlled and operated remotely over a wireless communication network that consists of the Internet of Things (IoT), short-range communication network, and long-range communication network.

13. A method for algal cultivation, said method comprises the following steps of:
providing a reservoir (1) having a flow path and an opening;
filling the reservoir (1) with the fluid medium up to a predetermined depth such that ambient light is incident on the fluid medium contained in the reservoir (1);
circulating the fluid medium along the flow path at a predetermined velocity;
at least partially covering said opening to at least partially block the light incident on the fluid medium and thereby define intermittent light and dark cycle for the fluid medium for a predetermined period of time in the reservoir (1) to define a predetermined ration of light and dark time incident to which the circulating fluid medium is exposed.

14. The method as claimed in claim 13, wherein said fluid medium is circulated at a velocity is in the range of 5 cm/s to 30 cm/s, preferably 5 cm/s to 15 cm/s, the depth of fluid medium is maintained between 5 cm and 30 cm, the temperature of the fluid medium is maintained between 28° C. and 40° C.

15. The method as claimed in claim 13, wherein the method comprises covering the opening with the help of a covering means such that the covered footprint area is in the range of 15% to 55%, preferably in the range of 28% to 40%, still preferably 36% and the light time to dark time ration in the range of 6:1, preferably 3:1.5, and still preferably 1.75:1.

16. The method as claimed in claim 13, being automated and further comprising the following steps of:
(vi) sensing, in the reservoir (1), the depth of the fluid medium, the temperature of the fluid medium, the velocity of the circulating medium in the flow path, and the amount of light incident on the fluid medium by means of a plurality of sensors;
(vii) maintaining the depth of the fluid medium to between 5 cm and 30 cm, maintaining the temperature of the fluid medium in the range of 28 C to 40 C, maintaining the velocity of the circulating medium in the range of 5 cm/s to 30 cm/s, preferably 5 cm/s to 15 cm/s;
(viii) covering the footprint area of the reservoir to the extent of 15% to 55%, preferably in the range of 28% to 40%, still preferably 36%;
(ix) maintaining a light to dark cycle for the fluid medium to lies is in the range of 6:1, preferably 3:1.5, and still preferably 1.75:1 to maintain a florescence ratio in the range of 0.5 to 0.7; and
(x) controlling the sensing, the circulating fluid medium and the operation of the covering by means of a processor and a memory in accordance with predetermined rules.

17. The method as claimed in claim 16, wherein the method is controlled remotely with the help of a wireless communication network that consists of the Internet of Things (IoT), short-range communication network, and long-range communication network.
